(19) United States
(12) Patent Application Publication
(10) Pub. No.: US 2024/0167108 A1
(43) Pub. Date: May 23, 2024

(54) NUCLEIC ACID TEMPLATE FOR DETECTION OF TARGET NUCLEIC ACID BASED ON G-QUADRUPLEX SEQUENCE AND USE THEREOF

(71) Applicant: PROGENEER INC., Seoul (KR)

(72) Inventors: Soong Ho UM, Seoul (KR); Sung OH, Seoul (KR); Jaehyun KANG, Seoul (KR); So Yeon AHN, Seoul (KR); Yelim KIM, Seoul (KR); Jeonghun KIM, Seoul (KR)

(21) Appl. No.: 18/552,186

(22) PCT Filed: Apr. 15, 2022
(86) PCT No.: PCT/KR2022/005463

§ 371 Date: Sep. 24, 2023

(30) Foreign Application Priority Data
Apr. 15, 2021 (KR) 10-2021-0049347

(51) Int. Cl. C12Q 1/70; C12Q 1/6839

(52) U.S. Cl. CPC C12Q 1/701; C12Q 1/6839

(57) ABSTRACT

The present invention relates to a nucleic acid template for detection of a target nucleic acid based on a G-quadruplex sequence and a use thereof. A nucleic acid template for detection of a target nucleic acid or a method for detection or diagnosis of a target nucleic acid, using same according to the present invention, allows for simple and rapid detection of a target nucleic acid at room temperature with high specificity through addition and reaction of a reaction enzyme even without a separate PCR machine or a complicated temperature control procedure. In addition, the template and the method exhibit high detection sensitivity because of taking advantage of rolling circle amplification or rolling circle transcription based on circular ring formation to amplify signals, and can instantly detect a target nucleic acid even without expensive signaling substances such as fluorescent molecules, or a separate signal detection procedure such as electrophoresis because of visibly forming a gel in the presence of the target nucleic acid. Therefore, the template and the method are useful in various fields such as infectious diseases, cancer diagnosis, hereditary disease, and customized diagnosis.

### TECHNICAL FIELD

The present invention relates to a nucleic acid template for detecting a target nucleic acid based on a G-quadruplex sequence and use thereof, and more particularly to a nucleic acid template for detecting a target nucleic acid including a sequence complementary to a G-quadruplex sequence and a first complementary sequence and a second complementary sequence complementary to a target nucleic acid, in which the first complementary sequence and the second complementary sequence are present at the 3′ end and the 5′ end of the nucleic acid template, a composition or diagnostic kit for detecting a nucleic acid including the same, and a method of diagnosing a disease such as COVID-19 using the same.

### BACKGROUND ART

A method of detecting a specific nucleic acid (such as DNA or RNA) is fundamentally important technology in the field of scientific research. By being able to detect and identify a specific nucleic acid, the presence of specific genes in samples, modification of genes, and the presence or absence of genetic and biological markers such as nucleic acids derived from specific pathogens may be confirmed, making it possible to predict the health status of a subject, diagnosis and prognosis of a specific disease, and response to a specific drug. Such molecular diagnosis diagnoses the root cause of diseases such as DNA or RNA, and is used in various fields such as diagnosis of infectious disease, cancer, and genetic disease, and customized diagnosis. As representative molecular diagnosis technology, there is PCR that amplifies DNA in a short time (Saiki, R., et al. Primer-directed enzymatic amplification of DNA with a thermostable DNA polymerase. Science 239, 487-91. 1998). However, general PCR, which necessarily involves 3 basic steps of denaturation→hybridization (primer binding)→nucleic acid synthesis, is typically performed by a machine, and requires electrophoresis to identify amplified DNA. Such electrophoresis is cumbersome because an agarose gel is prepared and DNA is identified through staining with EtBr or the like. In addition, real-time PCR that is recently proposed uses fluorescence, such that electrophoresis is unnecessary, but there is a problem in that expensive equipment and expensive fluorescent reagents must be used (Higuchi, R., et al., Kinetic PCR Analysis: Real-time Monitoring of DNA Amplification Reactions. Nature Biotechnology 11, 1026-1030, 1993). Recently, Cepheid's GeneXpert system and reagents, which are PCR products based on point-of-care diagnostics, have been developed and sold, but the system and reagents are very expensive and are difficult to use in general tests (Helb, D., et al., Rapid Detection of Mycobacterium tuberculosis and Rifampin Resistance by Use of On-Demand, Near-Patient Technology. J. Clin. Microbiol. 48, 229-237, 2010). Another method is a nucleic acid lateral flow assay, which uses a membrane instead of gel electrophoresis after PCR (Aveyard, J., et al., One step visual detection of PCR products with gold nanoparticles and a nucleic acid lateral flow (NALF) device. Chem. Commun., 41, 4251-4253, 2007). However, this method is more complicated than gel electrophoresis technology, making it impossible to perform the same in the laboratory, and limitations are also imposed on general use thereof due to technical difficulties in allowing the sequence of the probe attached to the membrane to specifically bind depending on the PCR amplification product. In particular, the PCR-based sequencing method is extremely vulnerable to certain errors and has frequent serious misdiagnosis due to problems in the data interpretation process, and thus representing, as fluorescence, potential difference, color, etc., the change when a specific nucleic acid biomarker in the blood responds to a sequence complementary thereto is under study. However, since most of these studies detect one biomarker at a time, it is difficult to effectively diagnose most diseases involving multiple biomarkers.

Meanwhile, coronavirus is a type of RNA virus, genetic information of which is composed of ribonucleic acid (RNA). Coronaviruses cause respiratory and gastrointestinal infections in humans and animals. Coronavirus is easily transmitted mainly by mucosal transmission, droplet transmission, etc., and generally causes mild respiratory infections in humans, but unusually causes fatal infections. Among coronavirus infections, severe acute respiratory syndrome-coronavirus (SARS-COV) broke out in China in April 2003, with a mortality rate of 9.6%, many people died, and in 2015, Middle East respiratory syndrome-coronavirus (MERS-CoV) spread from the Middle East to the world, showing a high mortality rate of about 36%, and since December 2019 the severe acute respiratory syndrome-coronavirus-2 (SARS-COV-2) epidemic from China is ongoing.

Severe acute respiratory syndrome-coronavirus-2 (SARS-COV-2) emerged in Wuhan, China and spread rapidly around the world, and the WHO named the disease due to infection with the relevant virus as COVID-19. According to a recent report, common symptoms include fever (98.6%), fatigue (69.6%), dry cough (59.4%), lymphopenia (70.3%), prolongation of prothrombin time (58%), and enhancement of lactate dehydrogenase (39.9%) (Wang, D., et al., JAMA, 2020). It has been reported that SARS-COV-2 is mainly transmitted through person-to-person contact via respiratory droplets generated by coughing and sneezing or through object surfaces contaminated by people who cough or sneeze (CDC, C.f.d.c.a.p., How COVID-19 Spreads. Coronavirus Disease 2019 (COVID-19), 2020), and it has even been reported that asymptomatic infection is possible (Yu, P., et al., J Infect Dis, 2020; Hoehl, S., et al., N Engl J Med, 2020; Bendix, A., Science alert, 2020).

As cases of SARS-COV-2 surged around the world, the WHO declared a “Public Health Emergency of International Concern” (PHEIC) on Jan. 30, 2020. As confirmed cases of coronavirus infections continued to rise around the world, the WHO on March 11 declared COVID-19 a pandemic (global pandemic) for the third time in history, following Hong Kong flu (1968) and swine flu (2009). As of Nov. 12, 2020, about 52 million patients occurred worldwide, about 1.3 million people died, and tens of thousands of confirmed patients were added every day, and the number of infected patients continues to increase. In Korea, about 28,000 patients have been confirmed and 487 deaths have been reported (COVID-19 Dashboard by the Center for Systems Science and Engineering (CSSE) at Johns Hopkins University (JHU) and Central Disaster and Safety Countermeasures Headquarters).

To date, there is no medical preventive measure for COVID-19 such as a vaccine, and active measures such as wearing a mask, washing hands, social distancing, and the like are being carried out at the personal/social level to prevent the spread of coronavirus. Although there is a slight decrease in the spread of infection, cases of infection are occurring in various regions, including churches, and the route thereof is unclear. Currently, 100 or more confirmed cases are occurring every day based on domestic standards, SO the demand for diagnosis of coronavirus is not decreasing. Therefore, since the supply of coronavirus diagnostics is still significantly insufficient relative to demand, the Guidelines for the Laboratory Diagnosis of 2019 novel coronavirus in Korea stipulate that the general public cannot receive a diagnostic test for COVID-19, and also that the test may be performed only for 4 specific indications (Korean Society for Laboratory Medicine, COVID-19 Task Force and the Center for Laboratory Control of Infectious Disease, the Korea Centers for Disease Control and Prevention, 2020). Therefore, it is necessary to develop technology capable of diagnosing COVID-19 inexpensively, simply/quickly, and with high accuracy that may meet the demand for diagnosis of COVID-19.

Against this background, the present inventors have made great efforts to develop a method capable of detecting a target nucleic acid quickly and accurately at low cost while being able to be performed at room temperature without complicated reaction procedures, and designed a nucleic acid template for detection of a target nucleic acid including a sequence complementary to a target gene and a sequence complementary to a G-quadruplex sequence and a detection protocol using the same, and thus ascertained that, based on results of detection of a target nucleic acid derived from SARS-COV-2 virus using the nucleic acid template and detection protocol, when the target nucleic acid is present, the nucleic acid template specifically binds to form a circular template, a nucleic acid is synthesized through rolling circle amplification or rolling circle transcription using the same as a template, and a nucleic acid gel is formed through a G-quadruplex structure, but such a nucleic acid gel is not formed in a similar sequence derived from SARS virus, making it possible to detect a target nucleic acid and diagnose a disease with high specificity and sensitivity without a separate detection procedure or signal material, thereby culminating in the present invention.

The above information described in the background section is only for improving the understanding of the background of the present invention, and it does not include information forming the prior art known to those of ordinary skill in the art to which the present invention pertains.

[Disclosure]

It is an object of the present invention to provide a nucleic acid template for detecting a target nucleic acid, including a sequence complementary to a G-quadruplex sequence, and use thereof.

It is another object of the present invention to provide a composition or kit for diagnosing a viral infection of a target nucleic acid, including the nucleic acid template.

It is still another object of the present invention to provide a kit for diagnosing a viral infection, including the nucleic acid template or composition.

It is yet another object of the present invention to provide a method of detecting a target nucleic acid using the nucleic acid template.

It is still yet another object of the present invention to provide a method of diagnosing a disease using the nucleic acid template.

It is a further object of the present invention to provide the use of the nucleic acid template for detecting a target nucleic acid or diagnosing a disease.

It is still a further object of the present invention to provide the use of the nucleic acid template for the manufacture of a composition for detecting a target nucleic acid or a composition or kit for diagnosing a disease.

In order to accomplish the above objects, the present invention provides a nucleic acid template for detecting a target nucleic acid, including a sequence complementary to a G-quadruplex sequence and a first complementary sequence and a second complementary sequence complementary to the target nucleic acid,

in which the first complementary sequence and the second complementary sequence are present at the 3′ end and the 5′ end of the nucleic acid template.

In addition, the present invention provides a composition or kit for detecting a target nucleic acid including the nucleic acid template.

In addition, the present invention provides a composition or kit for diagnosing a disease or infectious disease including the nucleic acid template.

In addition, the present invention provides a method of detecting a target nucleic acid, including:
(a) carrying out reaction by adding the nucleic acid template according to any one of claims 1 to 8 and a ligase to a sample;
(b) synthesizing a nucleic acid by adding a primer and a nucleic acid polymerase to the reaction result; and
(c) determining the presence or absence of a target nucleic acid based on whether or not a nucleic acid gel is formed.

In addition, the present invention provides a method of diagnosing a disease or a method of providing information for diagnosis, including:
(a) carrying out reaction by adding the nucleic acid template according to any one of claims 1 to 8 and a ligase to a sample isolated from a subject; and
(b) synthesizing a nucleic acid by adding a primer and a nucleic acid polymerase to the reaction result of step (a); and
(c) determining whether or not a gel is formed.

In addition, the present invention provides the use of the nucleic acid template for detecting a target nucleic acid or diagnosing a disease.

In addition, the present invention provides the use of the nucleic acid template for the manufacture of a composition for detecting a target nucleic acid or a composition or kit for diagnosing a disease.

### DESCRIPTION OF DRAWINGS

FIG. 1 schematically shows the structure of a nucleic acid template for detecting a target nucleic acid according to the present invention and a target nucleic acid detection protocol using the same.

FIG. 2 schematically shows formation of a nick by binding of the nucleic acid template for detecting a target nucleic acid according to the present invention and a target nucleic acid, in which, in the nucleic acid template for target nucleic acid detection, blue represents the first complementary sequence (5′ end) and the second complementary sequence (3′ end), purple represents the Poly T sequence, yellow represents the T7 promoter or primer binding site, and green represents the sequence complementary to a G-quadruplex sequence,

FIG. 2a showing that, when the target nucleic acid is present, a first complementary sequence and a second complementary sequence bind to form a nick, and

FIG. 2b showing that a nick cannot be formed when the target nucleic acid binds to a nucleic acid including a non-complementary portion;

FIG. 3 schematically shows a ligation process of the nicked nucleic acid template for detecting a target nucleic acid, FIG. 3a showing that the nick formed when the target nucleic acid is present may be ligated through ligase addition and reaction, and FIG. 3b showing that ligation by a ligase is impossible when a nick is not formed due to binding to a nucleic acid including a non-complementary portion;

FIG. 4 schematically shows a process of performing rolling circle amplification (RCA) or rolling circle transcription (RCT) using a nucleic acid polymerase after a complete circular template is formed by ligation, in which a primer is also added to a starting point for amplification or transcription, and rolling circle amplification or rolling circle transcription is successfully performed only when the target nucleic acid is present (FIG. 4a), and when the target nucleic acid is not present or binds to a nucleic acid including a non-complementary sequence, replication or transcription is terminated at the 3′ end and the G-quadruplex sequence is not synthesized;

FIG. 5 schematically shows a process of detecting SARS-COV-2 virus, which is a target nucleic acid, by distinguishing the same from SARS-COV-1 virus having a similar sequence using the nucleic acid template for detecting a target nucleic acid in an embodiment of the present invention;

FIG. 6 shows results of electrophoresis using a 1% agarose gel for a control nucleic acid (SARS-COV-1), a target nucleic acid (SARS-COV-2), a nucleic acid template, and a T7 primer, in which samples loaded in respective lanes are as follows: lane 1 is a 100-bp-sized dsDNA ladder (Promega), lane 2 is a 40-bp-sized synthesized control ssRNA (SARS-COV-1) nucleic acid (IDT), lane 3 is a 40-bp-sized synthesized target ssRNA (SARS-COV-2) nucleic acid (IDT), lane 4 is a 124-bp-sized synthesized ssDNA nucleic acid template (IDT), and lane 5 is a 22-bp-sized synthesized T7 primer (IDT);

FIG. 7 shows results of electrophoresis using a 1% agarose gel after reacting a control nucleic acid (SARS-CoV-1) and a target nucleic acid (SARS-COV-2) with a nucleic acid template (ligation by a ligase), in which samples loaded in respective lanes are as follows: lane 1 is a 100-bp-sized dsDNA ladder (Promega), lane 2 is a sample subjected to ligation by a ligase using a 40-bp-sized ssRNA control nucleic acid (SARS-COV-1) and a nucleic acid template, lane 3 is a sample subjected to ligation by a ligase using a 40-bp-sized ssRNA target nucleic acid (SARS-CoV-2) and a nucleic acid template, and lane 4 is a sample subjected to ligation by a ligase using only a nucleic acid template, in which, when the target nucleic acid is present, a circular nucleic acid template is formed by ligation using a ligase, indicating that the position of the band is different from that of on the the control based electrophoresis result;

FIG. 8 shows results of electrophoresis using a 1% agarose gel after rolling circle amplification (RCA) or rolling circle transcription (RCT) of each sample with a T7 polymerase (NEB), in which samples loaded in respective lanes are as follows: lane 1 is a 100-bp-sized dsDNA ladder (Promega), lane 2 is a sample subjected to rolling circle amplification (RCA) after ligation of a control nucleic acid (SARS-COV-1) and a nucleic acid template, lane 3 is a sample subjected to rolling circle amplification (RCA) after ligation of a target nucleic acid (SARS-COV-2) and a nucleic acid template, lane 4 is a sample subjected to rolling circle amplification (RCA) after ligation using only a nucleic acid template without a target nucleic acid, and lane 5 is a sample subjected to rolling circle amplification (RNA) without ligation of a target nucleic acid (SARS-COV-2) and a nucleic acid template, in which, when the target nucleic acid (SARS-COV-2) is present and binds to the nucleic acid template followed by ligation by a ligase, a circular nucleic acid template is formed and long-stranded RNA (about 300 to 2000 bp in length in the example of FIG. 8) is formed upon rolling circle amplification (RCA), as shown in the results of lane 3, and unlike lane 3, long-stranded RNA is not formed when the target nucleic acid is not present or when there is no ligation by a ligase even in the presence of the target nucleic acid (lanes 2, 4, and 5);

FIG. 9 shows a photograph of a sample subjected to rolling circle amplification (RCA) or rolling circle transcription (RCT) using a T7 polymerase (NEB) after ligation of a target nucleic acid (SARS-COV-2) and a nucleic acid template, indicating that the sample is present in the form of a gel and is not sucked into the tip; and

FIG. 10 shows results of UV imaging in fluorobox (Vivilber) after staining rolling circle amplification (RCA) or rolling circle transcription (RCT) samples with SYBR2 (Invitrogen), FIG. 10a showing photographs using a gray color filter, and FIG. 10b showing photographs using a rainbow color filter capable of representing the intensity of brightness, in which a, b, and c are as follows: a is a sample subjected to rolling circle amplification (RCA) using a T7 polymerase after ligation using a control nucleic acid (SARS-COV-1) and a nucleic acid template, b is a sample subjected to rolling circle amplification (RCA) using a T7 polymerase after ligation using a target nucleic acid (SARS-CoV-2) and a nucleic acid template, and c is a sample subjected to rolling circle amplification (RCA) using a T7 polymerase after ligation using only a nucleic acid template without a target nucleic acid, in which, in a, b, and c, a gel due to the G-quadruplex sequence is formed by the circular template only in the presence of the target nucleic acid, the transcribed RNA is enriched due to the gel, and after staining with SYBR2, light is intensively visible in UV.

### MODE FOR INVENTION

Unless otherwise defined, all technical and scientific terms used herein have the same meanings as typically understood by those skilled in the art to which the present invention belongs. In general, the nomenclature used herein is well known in the art and is typical.

A hydrogel nucleic acid production system based on rolling circle transcription (RCT) using a nucleic acid including a G-quadruplex motif sequence (a sequence complementary to a G-quadruplex sequence) as a template was invented and filed by the present inventors (Korean Patent Application No. 10-2019-0136416). Since the hydrogel nucleic acid production system enables hydrogelation of the synthesized hydrogel nucleic acid by forming a G-quadruplex structure, it is able to greatly increase the stability of RNA and is useful as platform technology capable of producing peptides or proteins therefrom.

The present inventors have made efforts to create new uses based on G-quadruplex sequence-containing templates and nucleic acid hydrogels, and newly designed a nucleic acid template for detecting a target nucleic acid including a sequence complementary to a target gene and a sequence complementary to a G-quadruplex sequence, and a target nucleic acid detection protocol using the same, and ascertained that the target nucleic acid may be detected with high specificity and sensitivity without a separate detection procedure or signal material through in-vitro isothermal reaction using the nucleic acid template.

In particular, in an embodiment of the present invention, specific detection of the RdRP gene of SARS-COV-2, which is a target nucleic acid, is achieved by distinguishing the RdRP gene sequence of SARS-COV-2 from the RdRP gene sequence of SARS virus having a very similar sequence, confirming that the nucleic acid template of the present invention may be a new platform useful for the diagnosis of infectious diseases such as SARS-COV-2, and furthermore, through detection of various mutations such as SNP, nucleic acid biomarkers, etc., new uses for various fields such as diagnosis and customized treatment have been created.

Accordingly, an aspect of the present invention pertains to a nucleic acid template for detecting a target nucleic acid, including a sequence complementary to a G-quadruplex sequence and a first complementary sequence and a second complementary sequence complementary to the target nucleic acid,

in which the first complementary sequence and the second complementary sequence are present at the 3′ end and the 5′ end of the nucleic acid template.

As used herein, the term “G-quadruplex” refers to a structure formed by vertically stacking two or more G-tetraplexes appearing in DNA sequences having high guanosine residue content. Here, the G-tetraplex is a unique cubic DNA structure in a quadruple-stranded form through hydrogen bonding by pairing four guanosine-rich DNA strands with each other, and is a quartet structure formed by associating four guanines through Hoogsteen hydrogen bonding. The G-tetraplex is very stable under various biochemical conditions and the orientation within the tetraplex is variable. In particular, the cation (mainly K+) in the center of the G-tetraplex contributes to maintaining the structure of the G-quadruplex more stably. The G-quadruplex is known to have various biological functions in telomere and promoter regions (Henderson E, et al., Telomeric DNA oligonucleotides form novel intramolecular structures containing guanine-guanine base pairs., Cell ((December 1987)). In the present invention, “G-quadruplex” may be used interchangeably with “G4”.

A G-quadruplex is mainly formed in guanine-rich sequence regions (Murat P, Balasubramanian S (April 2014)). However, a G-quadruplex is not formed in all guanine-rich sequence regions. A method of predicting the ability to form a G-quadruplex has been reported previously (Todd A K, Johnston M, Neidle S (2005)). For example, Todd A K, Johnston M, et al. reported d (G3+N1-7G3+N1-7G3+N1-7G3+) (in which the subscript is the number of bases, N is any base, and d is an integer of 1 or more, meaning repetition of the nucleotide sequence in parentheses) as a general pattern forming a G-quadruplex. In the present invention, the G-quadruplex may be formed by a sequence in which 3 or more consecutive guanines and 1 to 7 arbitrary sequences are repeated one or more times.

As used herein, the term “G-quadruplex sequence” refers to a sequence capable of forming a G-quadruplex, and a nucleic acid strand including the G-quadruplex sequence is able to form a G-quadruplex. The G-quadruplex sequence included in a nucleic acid strand transcribed using the nucleic acid template of the present invention as a template forms a G-quadruplex, leading to hydrogelation.

In the present invention, the G-quadruplex sequence may be a nucleic acid sequence in which 3 or more consecutive guanines and 1 to 7 arbitrary sequences are repeated one or more times, most preferably 5′-TAGGGTTAGGGT-3′ (SEQ ID NO: 5). In the present invention, when the nucleic acid constituting the G-quadruplex sequence is RNA, uracil (U) may be located at the position of thymine (T).

As used herein, the term “sequence complementary to a G-quadruplex sequence” refers to a sequence complementary to the G-quadruplex sequence, and any sequence capable of forming a G-quadruplex upon amplification or transcription using the same as a template.

In the present invention, preferably, the sequence complementary to the G-quadruplex sequence may be complementary to a nucleic acid sequence in which 3 or more consecutive guanines and 1 to 7 arbitrary sequences are repeated one or more times, and is most preferably 5′-ACCCTAACCCTA-3′ (SEQ ID NO: 6).

In an embodiment of the present invention, when a target nucleic acid was present, a nucleic acid synthesized using the nucleic acid template of the present invention as a template was designed to include a G-quadruplex sequence (5′-UAGGGUUAGGGU-3′) to form a G-quadruplex.

As used herein, the term “complementary” refers to having complementarity capable of selectively hybridizing to the above-described nucleic acid sequence under any specific hybridization or annealing conditions, and has a meaning encompassing both substantially complementary and perfectly complementary, and preferably means perfectly complementary.

As used herein, the term “hybridization” means that two single-stranded nucleic acids form a duplex structure by pairing complementary nucleotide sequences. In the present specification, “hybridization” may be used interchangeably with “complementarily binding”. Pairing of nucleotide sequences preferably includes A/T (U) and G/C according to the Watson-Crick model, but is not limited thereto. Hybridization may occur when complementarity between single-stranded nucleic acid sequences is perfect (perfect match) or even when some mismatch bases are present. The extent of complementarity necessary for hybridization may vary depending on hybridization conditions, and may be particularly controlled depending on temperature.

As used herein, the terms “first complementary sequence” and “second complementary sequence” refer to sequences complementary to a target nucleic acid. “First” and “second” used in the first complementary sequence and the second complementary sequence are used for convenience to distinguish sequences complementary to the target nucleic acid located at the 5′ end or the 3′ end of the nucleic acid template of the present invention and are not construed as limiting the scope of the present invention. In the present specification, the first complementary sequence may refer to a sequence complementary to the target nucleic acid located at the 5′ end of the nucleic acid template, and the second complementary sequence may refer to a sequence complementary to the target nucleic acid located at the 3′ end of the nucleic acid template, unless otherwise specified.

In the present invention, the first complementary sequence and the second complementary sequence are preferably complementary to different regions of the target nucleic acid.

In the present invention, the first complementary sequence and the second complementary sequence preferably complementarily bind to the sequence of the target nucleic acid.

In the present invention, the first complementary sequence and the second complementary sequence may complementarily bind to the sequence of the target nucleic acid to form a nick.

As used herein, the term “nick” refers to an unconnected gap between the 3′ end and the 5′ end of the nucleic acid template resulting from complementary binding of the first complementary sequence and the second complementary sequence to the target nucleic acid.

In the present invention, the first complementary sequence may complementarily bind to the target nucleic acid from the nick formation site toward the 5′ end of the target nucleic acid.

In the present invention, the second complementary sequence may complementarily bind to the target nucleic acid from the nick formation site toward the 3′ end of the target nucleic acid.

In the present invention, the first complementary sequence and the second complementary sequence may be designed to have appropriate lengths depending on the length of the target nucleic acid. Each of the first complementary sequence and the second complementary sequence preferably has a length of 15 bp or more, but is not limited thereto.

In the present invention, in order to form a nick by complementary binding to the target nucleic acid, the first complementary sequence and the second complementary sequence may complementarily bind to a continuous sequence of the target nucleic acid.

For example, as shown in FIG. 2, in the present invention, the first complementary sequence and the second complementary sequence present at the 3′ end and the 5′ end of the nucleic acid template may be hybridized with respective complementary portions of the target nucleic acid, and a nick may be included between the 5′ end and the 3′ end of the nucleic acid template.

For example, as in an embodiment of the present invention, when the RdRP gene (SEQ ID NO: 1) of SARS-COV-2 virus is a target nucleic acid, the first complementary sequence and the second complementary sequence may be respective sequences represented by SEQ ID NO: 7 and SEQ ID NO: 8, but are not limited thereto.

In the present invention, the nucleic acid template may complementarily bind to the target nucleic acid to form a circular nucleic acid template including a nick between the 5′ end and the 3′ end of the nucleic acid template.

In the present invention, the nucleic acid template may be characterized in that a circular nucleic acid template including a nick cannot be formed when the target nucleic acid is not present.

In the present invention, when the first complementary sequence and the second complementary sequence bind to non-continuous sequences of the target nucleic acid, there may be a single-stranded portion of the target nucleic acid composed of one or more nucleotides between binding sites of the target nucleic acid to the first complementary sequence and the second complementary sequence.

As such, through polymerase extension reaction, the single-stranded portion between binding sites of the target nucleic acid to the first complementary sequence and the second complementary sequence may be used as a template to extend the ends of the nucleic acid template of the present invention, thereby forming a nick.

In the present invention, as can be obviously understood, when the bound nucleic acid as shown in FIG. 2b or 3b is not complementary to the first complementary sequence and the second complementary sequence, even if the polymerase extension reaction is performed, no nick is formed between the 3′ end and the 5′ end of the nucleic acid template.

In the present invention, the nick of a circular nucleic acid template including a nick may be ligated by a ligase to form a complete circular nucleic acid template, and when replication or transcription is performed using the same as a template, replication and transcription are cyclically repeated without termination, so that rolling circle transcription (RCT) or rolling circle amplification (RCA) may be performed.

In the present invention, when the target nucleic acid is not present, a nick is not formed, and thus a circular nucleic acid template is not formed despite use of a ligase, and rolling circle transcription or rolling circle amplification is not performed.

In the present invention, when a complete circular nucleic acid template is formed by ligation of the nick and when rolling circle transcription (RCT) or rolling circle amplification (RCA) is performed, the synthesized nucleic acid may include a G-quadruplex sequence.

In the present invention, the nucleic acid synthesized through rolling circle transcription (RCT) or rolling circle amplification (RCA) may be hydrogelated by forming a G-quadruplex.

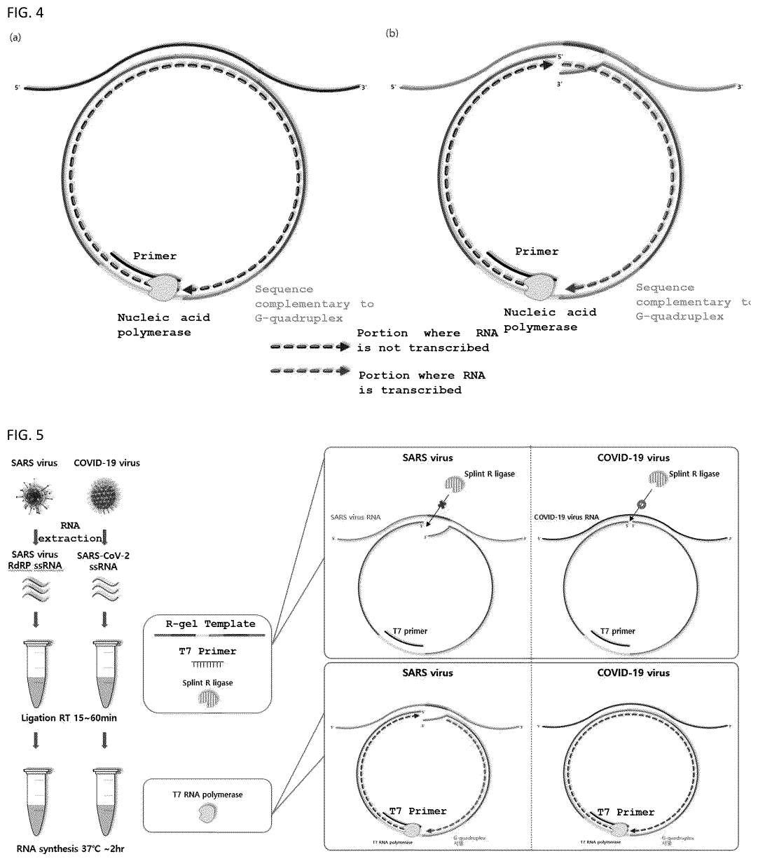

In an embodiment of the present invention, the nucleic acid template further includes a promoter sequence so that transcription using the nucleic acid template as a template is possible, and when the target nucleic acid is present, the nucleic acid template may form a circular nucleic acid template including a nick, followed by nick ligation using a ligase, leading to rolling circle transcription. On the other hand, when the target nucleic acid is not present, the nucleic acid template does not form a circular nucleic acid template including a nick, and transcription or replication is stopped at the 5′ end upon transcription or replication using the same as a template. As shown in FIG. 4, when the starting point of nucleic acid synthesis using the nucleic acid template as a template is present upstream (5′ direction) of the sequence complementary to the G-quadruplex sequence, the G-quadruplex sequence is not synthesized and the nucleic acid gel is not formed due to no synthesis at the 5′ end.

As in an embodiment of the present invention, when a nucleic acid is synthesized through transcription using the nucleic acid template of the present invention as a template, a promoter sequence may be required.

In the present invention, the nucleic acid template may further include a promoter sequence.

In the present invention, according to claim 1, the nucleic acid template may further include a promoter sequence upstream 15′ direction) of the sequence complementary to the G-quadruplex sequence.

As used herein, the term “promoter” refers to a gene region capable of regulating transcription initiation.

In the present invention, the promoter may be appropriately selected and used by those skilled in the art. Examples of the promoter may include T7 promoter, T5 promoter, T3 promoter, lac promoter, tac and trc promoters, cspA promoter, lacUV5 promoter, Ltet0-1 promoter, phoA promoter, araBAD promoter, trp promoter, tetA promoter, Ptac promoter, and SP6 promoter, and the promoter is preferably selected from among the examples above, but is not limited thereto.

As in an embodiment of the present invention, the nucleic acid template is most preferably configured such that the first complementary sequence complementary to the target nucleic acid, the promoter sequence, the sequence complementary to the G-quadruplex sequence, and the second complementary sequence complementary to the target nucleic acid are sequentially provided from the 5′ end to the 3′ end, but the present invention is not limited thereto.

In the present invention, the nucleic acid template may not include a promoter sequence.

In the present invention, when the nucleic acid template does not include a promoter sequence, it may include a primer binding site.

In the present invention, the primer binding site may be included upstream (5′ direction) of the G-quadruplex sequence.

In the present invention, the nucleic acid template not including the promoter sequence may serve to detect a target nucleic acid by performing replication (or amplification)-based nucleic acid synthesis using the same as a template. As such, the starting point of nucleic acid synthesis (replication, amplification) is preferably upstream of the G-quadruplex sequence, and the starting point of nucleic acid synthesis may be easily controlled by primer design or primer binding site design by those skilled in the art.

The nucleic acid template of the present invention may be constructed to a length sufficient to form a circular nucleic acid template including a nick. In the present invention, the length of the nucleic acid template is preferably at least 80 bp, more preferably 80 bp to 200 bp.

In an embodiment of the present invention, in order to provide sufficient flexibility to form a circular nucleic acid template, a Poly T sequence may be added between the first complementary sequence and the promoter sequence and between the sequence complementary to the G-quadruplex sequence and the second complementary sequence.

In the present invention, the nucleic acid template may further include a Poly T sequence.

In the present invention, the position where the Poly T sequence is included may be set without limitation except for the 5′ end or the 3′ end.

In the present invention, the Poly T sequence may be a 30-150 bp thymine (T) repeat sequence.

In the present invention, elements of the nucleic acid template (first complementary sequence, promoter sequence, sequence complementary to G-quadruplex sequence, Poly T, second complementary sequence, etc.) may be linked on a single linear nucleic acid template.

As used herein, the term “linkage” means that nucleic acids or nucleic acid strands having specific functions (first complementary sequence, promoter sequence, sequence complementary to G-quadruplex sequence, Poly T, second complementary sequence, etc.) are operably linked to each other. Here, “operably linked” refers to two components placed in a functional relationship with each other. Each linked sequence may be located upstream and/or downstream of each component. The linkage may be achieved by ligation at restriction sites. In cases where such sites do not exist, a synthetic oligonucleotide adapter or linker is used according to a typical practice. However, elements (first complementary sequence, promoter sequence, sequence complementary to G-quadruplex sequence, Poly T, second complementary sequence, etc.) need not be contiguous to be operably linked.

As used herein, the term “target nucleic acid” refers to a target (objective) for detecting the presence or absence thereof in a specific sample or a sample isolated from a subject using the nucleic acid template of the present invention.

In the present invention, the target nucleic acid may be DNA, RNA, PNA, LNA, etc., preferably DNA or RNA.

In the present invention, the target nucleic acid may be double-stranded or single-stranded, preferably single-stranded, but is not limited thereto.

In the present invention, the target nucleic acid may be selected without limitation according to the purpose of use of the nucleic acid template of the present invention. For example, the target nucleic acid may be a simple oligonucleic acid included in the sample, and depending on the purpose thereof, the target nucleic acid may be a genetic biomarker, a specific gene mutation such as SNP, deletion, etc., a gene derived from an infectious agent, or the like, but is not limited thereto.

In the present invention, the target nucleic acid may be a gene derived from an infectious agent.

In the present invention, the infectious agent may be a virus.

In the present invention, the virus may be a virus containing RNA as a genetic material, for example, SARS-CoV-1, SARS-COV-2, MERS virus, influenza virus, HIV, HCV, etc., but is not limited thereto.

Another aspect of the present invention pertains to the use of the nucleic acid template for detecting a target nucleic acid.

In the present invention, the target nucleic acid may be a biomarker of a specific disease, and thus may be used for various purposes such as prediction of diagnosis and prognosis of a specific disease, etc. Accordingly, still another aspect of the present invention pertains to the use of the nucleic acid template for diagnosing a disease.

Yet another aspect of the present invention pertains to the use of the nucleic acid template for the manufacture of a composition for detecting a target nucleic acid.

Still yet another aspect of the present invention pertains to the use of the nucleic acid template for the manufacture of a composition or kit for diagnosing a disease.

Even yet another aspect of the present invention pertains to a composition for detecting a target nucleic acid including the nucleic acid template of the present invention.

In the present invention, the composition may further include at least one selected from among a primer, a ligase, and a nucleic acid polymerase.

As used herein, the term “primer” refers to a short nucleic acid strand providing a 3′ end to synthesize a nucleic acid by performing transcription or replication using the nucleic acid template of the present invention as a template.

In the present invention, when the nucleic acid template includes a promoter sequence, the primer may include a sequence complementary to the promoter sequence.

In the present invention, when the nucleic acid template does not include a promoter sequence, the primer may bind upstream (5′-end) of the sequence complementary to the G-quadruplex sequence of the nucleic acid template.

As used herein, the term “ligase” refers to an enzyme that catalyzes the formation of phosphodiester bonds of nucleic acids to link/ligate nucleic acids. In the present invention, the ligase may be a ligase derived from various organisms or a variant thereof, and is preferably derived from E. coli, T4 bacteriophage, mammals, etc., and in an embodiment of the present invention, splint R ligase is used, but the present invention is not limited thereto. The ligase may be, for example, PBCV-1 DNA ligase, T4 DNA ligase, etc., depending on the type of nucleic acid constituting the template, but is not limited thereto.

As used herein, the term “nucleic acid polymerase” refers to an enzyme that catalyzes the synthesis of a nucleic acid monomer (DNA or RNA) complementary to the sequence of the template strand along a nucleic acid strand read as a template.

In the present invention, the nucleic acid polymerase may be selected and used depending on the type of nucleic acid to be synthesized, the type of promoter used, and the like.

In the present invention, when the nucleic acid template includes a promoter sequence, the nucleic acid polymerase may be an RNA polymerase. The RNA polymerase may be used for synthesis (transcription) of RNA using the nucleic acid template of the present invention as a template.

In the present invention, the RNA polymerase may be selected from the group consisting of, for example, RNA polymerases I to III, T7 RNA polymerase, and alpha-amanitin. In an embodiment of the present invention, T7 RNA polymerase is used, but the present invention is not limited thereto.

In the present invention, when the nucleic acid template does not include a promoter sequence, the nucleic acid polymerase may be a DNA polymerase. The DNA polymerase may be used for synthesis (replication/amplification) of DNA using the nucleic acid template of the present invention as a template.

In the present invention, the DNA polymerase is preferably DNA polymerase III, but is not limited thereto.

In the present invention, when the DNA polymerase is included, a coenzyme such as magnesium may be further included.

The nucleic acid template for detecting a target nucleic acid or the composition for detecting a target nucleic acid according to the present invention may be used in various medical fields such as prediction of diagnosis and prognosis of a specific disease, and prediction of response to a specific drug.

In particular, in an embodiment of the present invention, it was demonstrated that it is possible to specifically detect the SARS-COV-2 RdRP gene by distinguishing the genetic sequences of SARS virus and SARS-CoV-2 that are phylogenetically similar, and thus diagnosis of SARS-COV-2 infection (COVID-19) is possible.

Accordingly, a further aspect of the present invention pertains to a composition for diagnosing a disease including the nucleic acid template of the present invention or the composition for detecting a nucleic acid of the present invention.

Still a further aspect of the present invention pertains to a kit for diagnosing an infectious disease including the nucleic acid template of the present invention or the composition for detecting a nucleic acid of the present invention.

In the present invention, the diagnosis composition or kit may further include at least one selected from among a primer, a ligase, and a nucleic acid polymerase.

In the present invention, the disease is not limited so long as it may be diagnosed through detection of the target nucleic acid. In the present invention, the disease may be exemplified by an infectious disease, but is not limited thereto.

As used herein, the term “infectious disease (infection)” refers to a disease caused by infection of cells, tissues, organs, etc. by external pathogens such as various bacteria, spirochetes, rickettsia, viruses, fungi, and parasites. In the present invention, the diagnosis kit may be used without limitation for an infectious disease caused by a nucleic acid-containing pathogen, preferably an infectious disease caused by a virus.

In the present invention, the infectious disease may be, for example, a viral infection caused by SARS-COV-1, SARS-COV-2, MERS virus, influenza virus, HIV, HCV, etc., but is not limited thereto.

In the present invention, the infectious disease may be SARS-COV-2 infection (COVID-19).

In an embodiment of the present invention, a nucleic acid detection protocol including the steps of reaction by adding the nucleic acid template of the present invention and a ligase to a detection sample and then transcription by adding an RNA polymerase thereto was designed, and based on results confirming the reaction product at each step by targeting the RdRP gene of SARS-COV-2, as expected, only when the target nucleic acid is present, the nucleic acid template and the target nucleic acid complementarily bind to form a circular nucleic acid template including a nick, the nick is linked by a ligase, and RCT is successfully performed, thereby forming a nucleic acid gel.

Therefore, yet a further of the present invention pertains to a method of detecting a target nucleic acid, including:
(a) carrying out reaction by adding the nucleic acid template of the present invention and a ligase to a sample;
(b) synthesizing a nucleic acid by adding a primer and a nucleic acid polymerase to the reaction result; and
(c) determining the presence or absence of a target nucleic acid based on whether or not a nucleic acid gel is formed.

In the present invention, in step (a), when the target nucleic acid is present in the sample, sequences complementary to the target nucleic acid present at the 5′ and 3′ ends of the nucleic acid template of the present invention are hybridized with the target nucleic acid, so that a circular nucleic acid template including a nick may be formed.

In the present invention, in step (a), when the target nucleic acid is not present, a circular nucleic acid template may not be formed.

In the present invention, in step (a), when there is a similar sequence that is partially different from the target nucleic acid, even if some complementary portions are hybridized, the non-complementary portion is not hybridized and a nick is not formed.

In the present invention, in step (a), when the target nucleic acid is present and a circular nucleic acid template including a nick is formed, the 3′ end and the 5′ end of the nucleic acid template are linked by a ligase, forming a complete circular nucleic acid template.

In the present invention, in step (a), when the target nucleic acid is not present, there is no nick, and thus the 5′ end and the 3′ end are not linked by a ligase, so that a complete circular nucleic acid template is not formed.

In the present invention, the nucleic acid synthesized in step (b) may be DNA or RNA.

In the present invention, in step (b), when the target nucleic acid is present in the sample, rolling circle transcription (RCT) or rolling circle amplification (RCA) may be performed using the nucleic acid template of the present invention as a template.

In the present invention, in step (b), when the target nucleic acid is present, a sequence including a G-quadruplex sequence may be synthesized.

In the present invention, in step (b), when the target nucleic acid is present in the sample, a nucleic acid gel may be formed.

In the present invention, step (c) may further include determining that the target nucleic acid is present in the sample when the nucleic acid gel is formed or determining that the target nucleic acid is not present in the sample when the nucleic acid gel is not formed.

As used herein, the term “sample” refers to a target sample for which the presence or absence of the target nucleic acid is to be confirmed through the method of the present invention.

As used herein, the term “nucleic acid gel” refers to a nucleic acid that forms a gel through various interactions such as hydrogen bonding, covalent bonding, hydrophobic interaction, etc. between nucleic acid sequences. In particular, the present invention is characterized in that the G-quadruplex sequence forms a unique cubic structure in a quadruple-stranded form through hydrogen bonding, yielding a gel. In order to solve the problems with the above RNA synthesis method and synthesized RNA, the present inventors invented and filed a hydrogel nucleic acid production system based on rolling circle transcription (RCT) using a nucleic acid including a G-quadruplex motif sequence as a template (Korean Patent Application No. 10-2019-0136416). In the present invention, “nucleic acid gel” is used interchangeably with “hydrogel nucleic acid” or “nucleic acid hydrogel”.

In the present invention, steps (a) and (b) may be performed simultaneously or sequentially, and when performed simultaneously, reaction may be carried out by adding the nucleic acid template of the present invention, ligase, primer, and nucleic acid polymerase to the sample.

The method of detecting the target nucleic acid according to the present invention may be useful in the medical field such as diagnosis through detection of biomarkers, genes derived from infectious agents, and the like.

Therefore, yet a further aspect of the present invention pertains to a method of providing information for diagnosis of a disease, including:
(a) carrying out reaction by adding the nucleic acid template according to any one of claims 1 to 8 and a ligase to a sample isolated from a subject;
(b) synthesizing a nucleic acid by adding a primer and a nucleic acid polymerase to the reaction result of step (a); and
(c) determining whether or not a gel is formed.

Still yet a further aspect of the present invention pertains to a method of diagnosing a disease, including:
(a) carrying out reaction by adding the nucleic acid template according to any one of claims 1 to 8 and a ligase to a sample isolated from a subject;
(b) synthesizing a nucleic acid by adding a primer and a nucleic acid polymerase to the reaction result of step (a); and
(c) determining whether or not a gel is formed.

In the diagnosis method or information provision method for diagnosis according to the present invention, individual steps may share the same characteristics as the corresponding steps in the method of detecting the target nucleic acid.

As used herein, the term “subject” refers to a subject diagnosed as suffering from a disease or not. The subject means not only animals, but also all subjects such as cells, tissues, and organs. Preferably the subject is a human.

The present invention is preferably used for the diagnosis of human coronavirus infection, particularly COVID-19, and may be used for the prediction of diagnosis and prognosis of various diseases and the prediction of drug response through the detection of biomarkers using the nucleic acid template, composition, kit, or method of the present invention, or used for the diagnosis of infection by detecting RNA of viruses or bacteria other than coronavirus, as will be obvious to those skilled in the art.

In the present invention, any sample may be used so long as it contains a nucleic acid or gene derived from a subject to be diagnosed, and the nucleic acid or gene may be DNA or RNA, and the sample derived from a subject indicates whole or part of the patient's body or secretions isolated from the patient, such as blood, tissue sample, feces, urine, sputum, etc. It is preferable not to return the whole or part of the patient's body or secretions isolated from the patient to the patient's body.

A better understanding of the present invention may be obtained through the following examples. These examples are merely set forth to illustrate the present invention, and are not to be construed as limiting the scope of the present invention, as will be obvious to those skilled in the art.

### Example 1: Design of Nucleic Acid Template for Target Nucleic Acid Detection

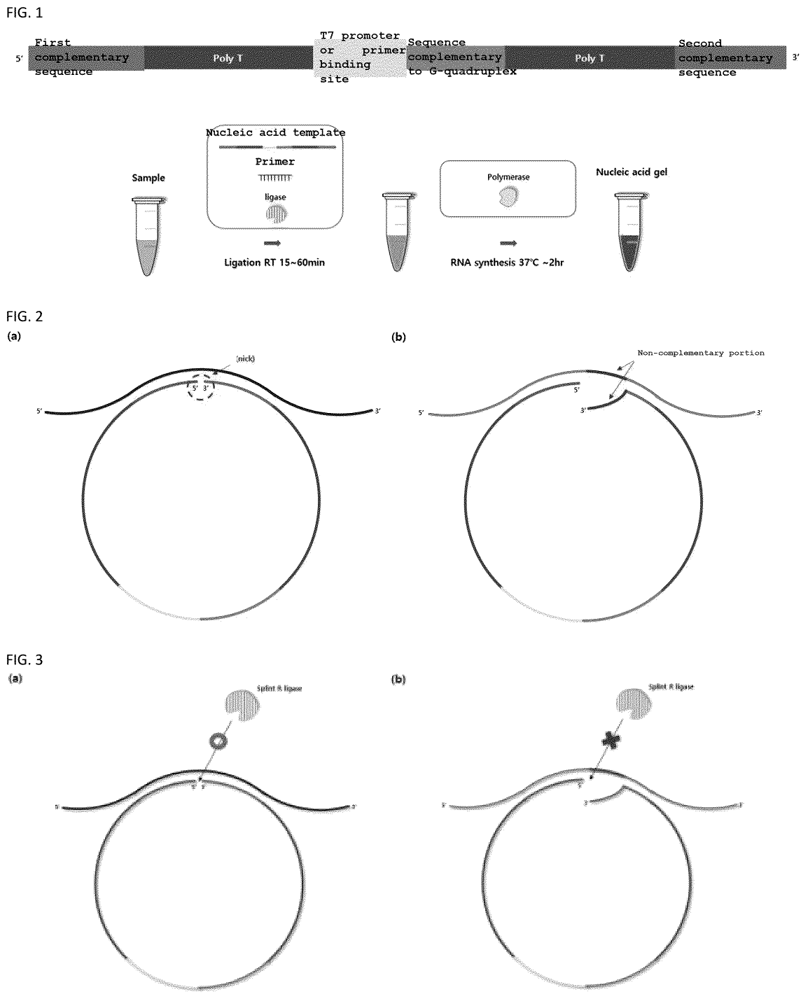

In order to develop a nucleic acid template based on a G-quadruplex sequence capable of detecting a target nucleic acid, a nucleic acid template including a first complementary sequence complementary to the target nucleic acid, a sequence complementary to a G-quadruplex sequence, and a second complementary sequence complementary to the target nucleic acid was designed (FIG. 1). A Poly T sequence may be added to each of the 5′ direction of the promoter sequence and the 3′ direction of the sequence complementary to the G-quadruplex sequence to impart the nucleic acid template with sufficient flexibility to form a circular loop. When adding a Poly T sequence, it is preferable to add a Poly T sequence with a length of 30 to 150 bp, but the present invention is not limited thereto.

The target nucleic acid detection protocol using the nucleic acid template of the present invention basically includes i) carrying out reaction by adding a nucleic acid template, a ligase, and a primer to a sample and ii) synthesizing a nucleic acid, in which some procedures during steps may be simultaneously performed (bottom of FIG. 1).

### Example 2: Target Nucleic Acid Detection Mechanism

i) In carrying out reaction by adding the nucleic acid template, the ligase, and the primer to the sample,

when the target nucleic acid is present in the sample, sequences complementary to the target nucleic acid present at the 5′ end and the 3′ end of the nucleic acid template of the present invention are hybridized with the target nucleic acid, forming a circular nucleic acid template including a nick. (FIG. 2a).

When the target nucleic acid is not present, a circular nucleic acid template is not formed, and when there is a similar sequence that is partially different from the target nucleic acid, even if some complementary portions are hybridized, the non-complementary portion is not hybridized and a nick is not formed (FIG. 2b).

When the target nucleic acid is present and a circular nucleic acid template including a nick is formed, the 3′ end and the 5′ end of the nucleic acid template are linked by a ligase, forming a complete circular nucleic acid template (FIG. 3a).

On the other hand, when the target nucleic acid is not present or even when a similar nucleic acid sequence is present, there is no nick, and thus the 5′ end and the 3′ end are not linked by a ligase, not forming a complete circular nucleic acid template (FIG. 3b).
ii) In synthesizing the nucleic acid,

when a nucleic acid polymerase is added and allowed to react, rolling circle transcription (RCT) or rolling circle amplification (RCA) is performed using the nucleic acid template of the present invention as a template. It is possible to visually detect the presence or absence of the target nucleic acid in the sample depending on whether or not the gel of the synthesized product through RCT or RCA is formed.

When the nucleic acid is synthesized using rolling circle transcription (RCT), transcription starts from the promoter sequence included in the nucleic acid template, and the primer sequence includes a sequence complementary to part or all of the promoter sequence. Here, the sequence complementary to the G-quadruplex sequence of the nucleic acid template is located downstream (3′ direction) of the promoter sequence.

When the target nucleic acid is present, a complete circular loop is formed during ligase reaction, enabling continuous RNA synthesis by rolling circle transcription (RCT). When rolling circle transcription is completely performed, the synthesized RNA includes a G-quadruplex sequence, and the G-quadruplex sequence forms a G-quadruplex structure, leading to hydrogelation (FIG. 4a). On the other hand, when the target nucleic acid is not present, a complete circular nucleic acid template is not formed, and when transcription starts, transcription is terminated at the 5′ end, so rolling circle transcription (RCT) is not completely performed, and the transcription product does not include a G-quadruplex sequence and therefore a gel is not formed (FIG. 4b).

When the nucleic acid is synthesized using rolling circle amplification (RCA), the nucleic acid template may not include a promoter sequence. Here, the primer sequence is designed to bind upstream (5′ direction) of the sequence complementary to the G-quadruplex sequence, and when the target nucleic acid is present, rolling circle amplification (RCA) is performed using a complete circular nucleic acid template as a template, and the synthesized nucleic acid is hydrogelated by the G-quadruplex sequence, whereas when the target nucleic acid is not present, a complete circular nucleic acid template is not formed (FIG. 4a), and when replication starts, synthesis is terminated at the 5′ end, so rolling circle amplification (RCA) is not completely performed, and the product does not include a G-quadruplex sequence and therefore a gel is not formed (FIG. 4b).

### Example 3: Specific Detection of SARS-COV-2 Gene Using Nucleic Acid Template

### Example 3-1: Construction of nucleic acid template for detection of SARS-COV-2 gene

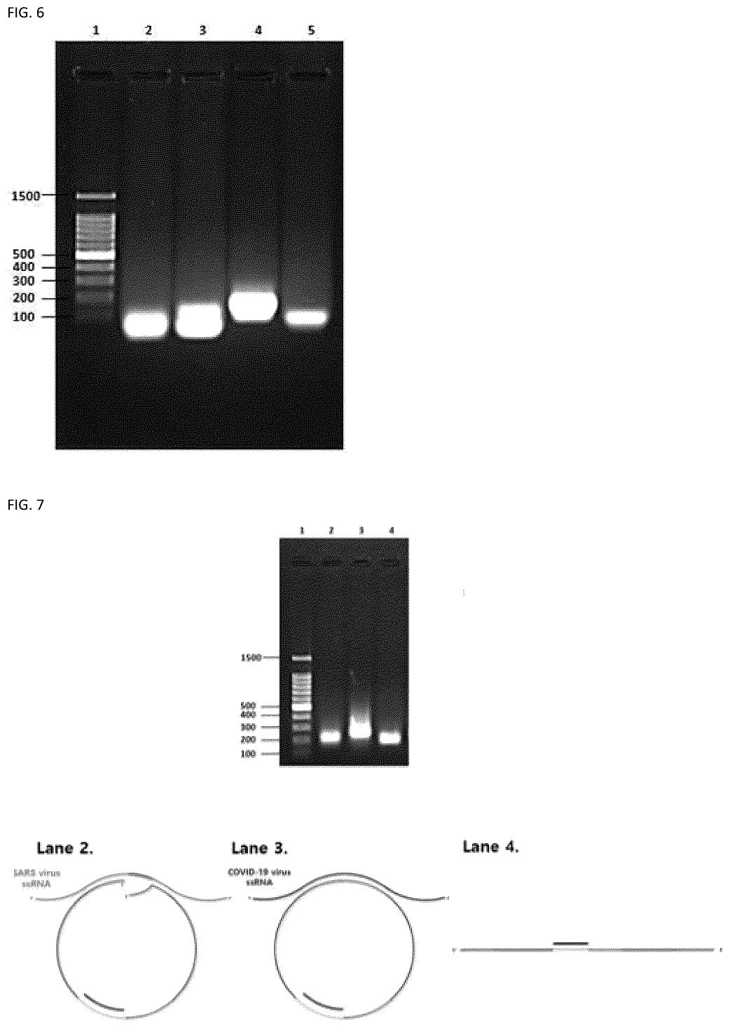

For verification of the designed nucleic acid template and detection protocol, the RdRP gene (gene number: NC 045512.2, 14093-14132, size: 40 bp) of SARS-COV-2, which has recently been most in diagnostic demand and of high interest, was selected as the target nucleic acid, and in order to confirm that it is possible to specifically detect the SARS-COV-2 gene, as a positive control, the RdRP gene (gene number: NC_004718.3, 14023-14062, size: 40 bp) derived from SARS virus (SARS-COV-1) very similar to that of SARS-CoV-2 was selected (FIG. 5). Each target nucleic acid (RNA oligomer), T7 primer, and nucleic acid template were artificially synthesized (integrated DNA technology), and the sizes thereof were confirmed through 18 agarose gel electrophoresis (FIG. 6). Lane 1 is a 100-bp-sized dsDNA ladder (Promega), lane 2 is a 40-bp-sized ssRNA control nucleic acid (SARS-COV-1), and lane 3 is a 40-bp-sized ssRNA target nucleic acid (SARS-COV-2). Lane 4 is a 124-bp-sized SSDNA nucleic acid template, and lane 5 is a 22-bp-sized SSDNA T7 primer. The reason why the band position of the 22-bp-sized ssDNA T7 primer (lane 5) is higher than that of the 40-bp-sized ssRNA (lanes 2 and 3) is that the speed of SSRNA is higher than that of ssDNA on agarose gel electrophoresis.

TABLE 1

| | Nucleic acid sequence (5′→3′) | SEQ ID NO: |
|---|---|---|
| Target nucleic | AUUUCGGUGAUUUCAUACAA/ACCACGCCAGGUAGUGGAGU | 1 |
| acid (SARS- | | |
| CoV-2) | | |
| Control (SARS- | AUUUCGGUGAUUUCGUACAA/GUAGCACCAGGCUGCGGAGU | 2 |
| CoV-1) | | |
| Nucleic acid | TTGTATGAAATCACCTTTTTTTTTTTTTTTTTTTTTTTTTTT | 3 |
| template | TTTATCCCTATAGTGAGTCGTATTAACCCTAACCCTATTT | |
| | TTTTTTTTTTTTTTTTTTTTTTTTTTTACTACCTGGCGTGGT | |
| T7 primer | TAATACGACTCACTATAGGGAT | 4 |

*Underlines of nucleic acid template sequence indicate portions complementary to a target nucleic acid (first and second complementary sequences) .
* ″/″ in target nucleic acid and control means a position where a nick is formed by binding of first and second complementary sequences of a nucleic acid template
*Bold means a sequence complementary to G-quadruplex sequence, and italics mean a promoter sequence

TABLE 2
Construction of nucleic acid template for detection of RdRP
gene of SARS-CoV-2

| | Nucleic acid sequence | |
|---|---|---|
| | (5′→3′) | SEQ ID NO: |
| G-quadruplex sequence | TAGGGTTAGGGT | 5 |
| Sequence complementary to G- | ACCCTAACCCTA | 6 |
| quadruplex sequence | | |
| First complementary sequence | TTGTATGAAATCACC | 7 |
| Second complementary sequence | ACTACCTGGCGTGGT | 8 |
| T7 promoter sequence | ATCCCTATAGTGAGTCGTATTA | 9 |

*In G-quadruplex sequence, T may be substituted with U

### Example 3-2: Confirmation of Formation of Target Nucleic Acid-Specific Circular Nucleic Acid Template

SARS-COV-2 RdRP ssRNA and control (SARS virus RdRP SSRNA) samples were mixed with the nucleic acid template constructed in Example 3-1, T7 primer, and splint R ligase, and allowed to react at room temperature for about 60 minutes. After completion of reaction, the mixtures were analyzed through 1% agarose gel electrophoresis.

As shown in FIG. 7, upon reaction with the RdRP gene of SARS-COV-2, which is the target nucleic acid (lane 3: SARS-COV-2), the 5′ end and the 3′ end of the nucleic acid template were ligated by a ligase to form a circular template, so that bands were observed at positions where the size was higher than the electrophoretic bands of the controls (lane 2: SARS-COV-1 and lane 4: D.W). As expected, the reaction proceeded only in the sample containing the RdRP gene of SARS-COV-2, which is the target nucleic acid, forming a complete circular nucleic acid template, and the SARS virus gene having a similar sequence or negative control (D.W) did not form a complete circular nucleic acid template.

### Example 3-3: Confirmation of Target Nucleic Acid-Specific Detection Ability

RNA synthesis was conducted by adding a T7 RNA polymerase (New England BioLabs) to the mixture reacted in Example 3-2 and performing incubation at 37ºC for about 2 hours. When a complete circular nucleic acid template is formed, the transcript is amplified through rolling circle transcription (RCT).

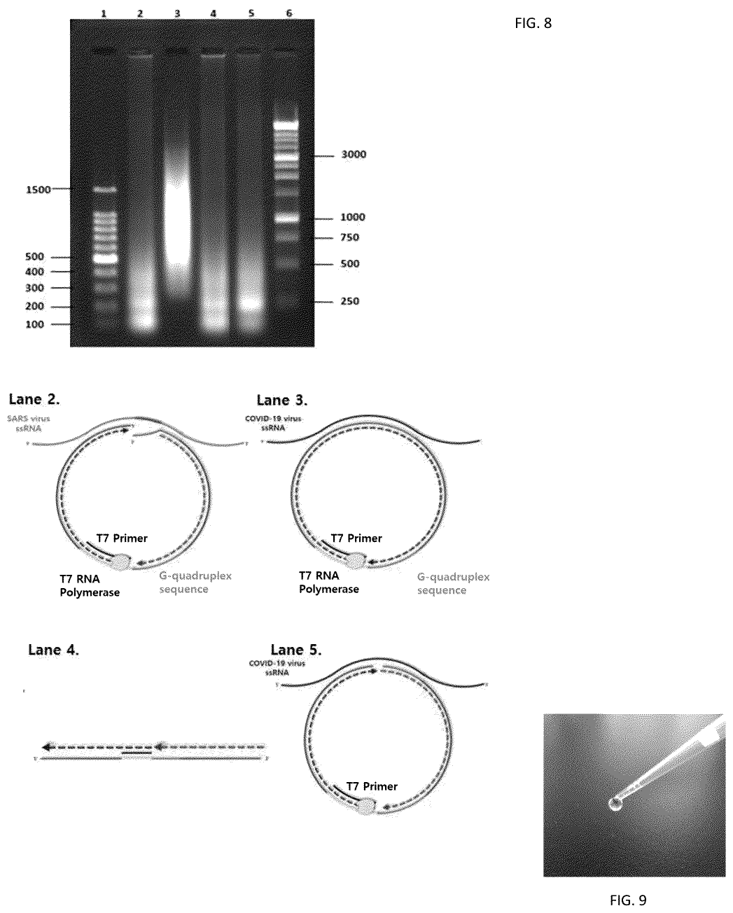

The solution subjected to RNA synthesis was analyzed through 1% agarose gel electrophoresis. As shown in FIG. 8, a wide band with a large size of about 500 to 2000 bp appeared only in the sample (lane 3) containing the RdRP RNA of SARS-COV-2, and for lane 5 in which a ligase was not added, rolling circle transcription was not performed, and only a low-sized band was observed. This means that a G-quadruplex is formed by a G-quadruplex sequence through amplification after target nucleic acid-specific rolling transfer transcription using the nucleic acid template of the present invention, resulting in a large complex.

### Examples 3-4: Confirmation of Formation of Target Nucleic Acid-Specific Nucleic Acid Hydrogel

RNA was synthesized by performing transcription in the reaction mixture including the nucleic acid template, the ligase, and the primer as in Example 3-3, and whether or not a hydrogel was formed was visually confirmed.

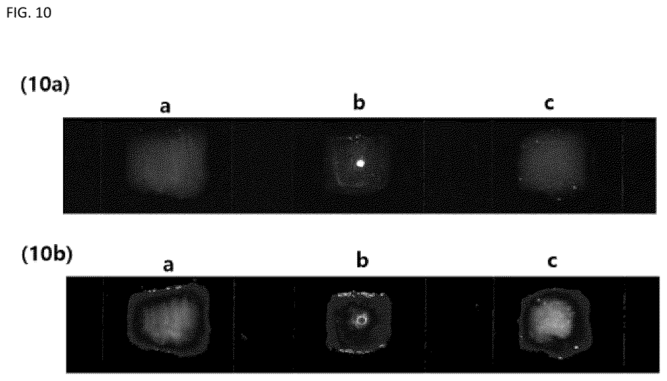

As shown in FIG. 9, an RNA transcript in the form of a hydrogel was confirmed in the RdRP RNA sample of SARS-CoV-2, allowed to react with PEG (polyethylene glycol), and treated with gel doc under white and blue light, based on which a gel was observed in the RdRP RNA sample of SARS-CoV-2 (white light: bright white, blue light: red), but a gel was not observed in the RdRP RNA sample of SARS virus and negative control (D.W) (FIG. 10).

Therefore, the target nucleic acid detection protocol using the nucleic acid template of the present invention is capable of forming a gel very specific to the target nucleic acid, which means that the target nucleic acid can be visually detected without a separate signal molecule or procedure.

### INDUSTRIAL APPLICABILITY

According to the present invention, a nucleic acid template for detecting a target nucleic acid and a method of detecting a target nucleic acid or a diagnosis method using the same are capable of detecting a target nucleic acid with high specificity simply and quickly by enzyme addition and reaction at room temperature without a separate PCR machine or a complicated temperature control procedure. In addition, rolling circle amplification or rolling circle transcription by formation of a circular loop results in signal amplification occurs and high detection sensitivity, and since a gel is visually formed in the presence of the target nucleic acid, the target nucleic acid can be immediately detected without expensive signal materials such as fluorescent molecules or separate signal detection procedures such as electrophoresis, and therefore, the present invention is useful in various fields such as diagnosis of infectious disease, cancer, and genetic disease, and customized diagnosis.

Having described specific parts of the present invention in detail above, it will be obvious to those skilled in the art that these specific descriptions are only preferred embodiments, and the scope of the present invention is not limited thereby. Accordingly, the substantial scope of the present invention will be defined by the appended claims and equivalents thereto.

### [Sequence List Free Text]

An electronic file is attached.

1. A nucleic acid template for detecting a target nucleic acid, comprising a sequence complementary to a G-quadruplex sequence; and a first complementary sequence and a second complementary sequence complementary to the target nucleic acid, wherein the first complementary sequence and the second complementary sequence are present at a 3′ end and a 5′ end of the nucleic acid template.

2. The nucleic acid template according to claim 1, wherein the nucleic acid template complementarily binds to the target nucleic acid to form a circular nucleic acid template including a nick between the 5′ end and the 3′ end of the nucleic acid template.

3. The nucleic acid template according to claim 1, further comprising a promoter sequence upstream (5′ direction) of the sequence complementary to the G-quadruplex sequence.

4. The nucleic acid template according to claim 3, wherein the nucleic acid template is configured such that the first complementary sequence complementary to the target nucleic acid, the promoter sequence, the sequence complementary to the G-quadruplex sequence, and the second complementary sequence complementary to the target nucleic acid are sequentially provided from the 5′ end to the 3′ end.

5. The nucleic acid template according to claim 1, wherein the nucleic acid template has a length of 80 to 200 bp.

6. The nucleic acid template according to claim 1, further comprising a Poly T sequence.

7. The nucleic acid template according to claim 1, wherein the target nucleic acid is a nucleic acid derived from SARS-COV-2 virus.

8. The nucleic acid template according to claim 7, wherein the nucleic acid template comprises the first complementary sequence represented by SEQ ID NO: 7 and the second complementary sequence represented by SEQ ID NO: 8.

9. A composition for detecting a target nucleic acid, comprising the nucleic acid template according to claim 1.

10. The composition according to claim 9, further comprising at least one selected from among a primer, a ligase, and a nucleic acid polymerase.

11. A kit for diagnosing a disease, comprising the nucleic acid template according to claim 1.

12. The kit according to claim 11, further comprising at least one selected from among a primer, a ligase, and a nucleic acid polymerase.

13. The kit according to claim 11, wherein the disease is SARS-COV-2 virus infection (COVID-19).

14. A method of detecting a target nucleic acid, comprising:
(a) carrying out reaction by adding the nucleic acid template according to claim 1, and a ligase to a sample:
(b) synthesizing a nucleic acid by adding a primer and a nucleic acid polymerase to a reaction result; and
(c) determining presence or absence of a target nucleic acid based on whether a nucleic acid gel is formed.

15. The method according to claim 14, wherein in step (a), when the target nucleic acid is present, the nucleic acid template binds to the target nucleic acid to form a circular nucleic acid template comprising a nick between a 5′ end and a 3′ end of the nucleic acid template.

16. The method according to claim 14, wherein in step (a), a nick of a circular nucleic acid template is linked by the ligase.

17. The method according to claim 14, wherein in step (b), when the target nucleic acid is present, a sequence comprising a G-quadruplex sequence is synthesized.

18. A method of diagnosing a disease, comprising:
(a) carrying out reaction by adding the nucleic acid template according to claim 1, and a ligase to a sample isolated from a subject;
(b) synthesizing a nucleic acid by adding a primer and a nucleic acid polymerase to a reaction result of step (a); and
(c) determining whether a gel is formed.

19. The method according to claim 18, wherein the disease is COVID-19.


SEQUENCE LISTING (9 sequences)

SEQ ID NO: 1 - RNA, 40 nt, Artificial Sequence
  Target Nucleic acid (SARS-CoV-2 RdRP)
auuucggugauuucauacaaaccacgccagguaguggagu

SEQ ID NO: 2 - RNA, 40 nt, Artificial Sequence
  SARS-CoV-1 RdRP
auuucggugauuucguacaaguagcaccaggcugcggagu

SEQ ID NO: 3 - DNA, 124 bp, Artificial Sequence
  Nucleic acid Template
ttgtatgaaatcaccttttttttttttttttttttttttttttttatccctatagtgagtcgtattaaccctaaccctattttttttttttttttttttttttttttttactacctggcgtggt

SEQ ID NO: 4 - DNA, 22 bp, Artificial Sequence
  T7 primer
taatacgactcactatagggat

SEQ ID NO: 5 - DNA, 12 bp, Artificial Sequence
  G-Quadruplex sequnce
tagggttagggt

SEQ ID NO: 6 - DNA, 12 bp, Artificial Sequence
  Complementary sequence of G-Quadruplex sequnce
accctaacccta

SEQ ID NO: 7 - DNA, 15 bp, Artificial Sequence
  the first complementary sequence
ttgtatgaaatcacc

SEQ ID NO: 8 - DNA, 15 bp, Artificial Sequence
  the second complementary sequence
actacctggcgtggt

SEQ ID NO: 9 - DNA, 22 bp, Artificial Sequence
  T7 promoter
atccctatagtgagtcgtatta